(19) United States
(12) Patent Application Publication
(10) Pub. No.: US 2023/0398066 A1
(43) Pub. Date: Dec. 14, 2023

(54) DRUG DOSAGE FORMS FOR STOMACH RETENTION

(71) Applicant: Triastek, Inc., Nanjing (CN)

(72) Inventors: Feihuang DENG, Nanjing (CN); Yu ZHENG, Nanjing (CN); Xiaoling LI, Dublin, CA (US); Senping CHENG, Nanjing (CN)

(21) Appl. No.: 18/034,004

(22) PCT Filed: Nov. 1, 2021
(86) PCT No.: PCT/CN2021/127852

§ 371 Date: Apr. 26, 2023

(30) Foreign Application Priority Data
Oct. 30, 2020 (WO) PCT/CN2020/125333

(51) Int. Cl. A61K 9/00; A61K 9/48; B33Y 80/00; B33Y 10/00

(52) U.S. Cl. CPC A61K 9/0065; A61K 9/4808; B33Y 80/00; B33Y 10/00

(57) ABSTRACT

Provided herein are drug dosage forms having a swellable component, which alone or in conjugation with one or more other components of the drug dosage forms expand the overall size of the drug dosage form in the stomach. The drug dosage forms are configured to be retained in the stomach for an extended period of time based on this increase in size of the drug dosage form in the stomach (e.g., retained in the stomach for a period of at least about 24 hours). The drug dosage forms described herein are formulated to release a drug during at least a portion of the stomach residency period. In other aspects, also provided are methods of designing, methods of making, such as using three-dimensional printing, and methods of delivering a drug to an individual, wherein such methods are associated with the drug dosage forms described herein.

### TECHNICAL FIELD

The present disclosure, in some aspects, is directed to drug dosage forms having a swellable component, wherein the swellable component is configured to swell in the stomach to increase the size of the drug dosage form such that the drug dosage form is retained in the stomach for an extended period of time. In other aspects, the present disclosure is directed to methods of designing, methods of making, such as using three-dimensional (3D) printing, and methods of delivering a drug to an individual, wherein such methods are associated with the stomach retentive drug dosage forms described herein.

### BACKGROUND

Conventional oral drug dosage forms, when administered to an individual, are subject to the natural flow of fluids, semi-solids, and solids through the gastrointestinal system of the individual. This natural flow can vary between administrations of a drug dosage form to a specific individual, and across a population of individuals, based on fluctuating circumstances relative to when the drug dosage form was administered, such timing and size of a meal and/or drink, the meal and/or drink contents, and the current stomach phase and remaining duration. These circumstance create variability in, e.g., the anatomical location of drug delivery, bioavailability, safety profile, and efficacy of a drug dosage form that has a period of residency in the stomach, such as an oral drug dosage form.

All references cited herein, including patent applications and publications, are incorporated herein by reference in their entirety.

### BRIEF SUMMARY

Provided herein, in some aspects, is a drug dosage form comprising a first component and a swellable component, wherein the first component and the swellable component are connected, wherein at least one of the first component and the swellable component comprises a drug, wherein the swellable component, or a portion thereof, swells upon exposure to gastrointestinal fluid, and wherein the swelling of the swellable component, or the portion thereof, increases the dimension of the drug dosage form, thereby allowing the drug to be retained in the stomach for at least about 24 hours.

In some embodiments, the swellable component comprises an exterior lid and a swelling agent, wherein the exterior lid is connected to the swelling agent, and wherein the exterior lid is configured such that it forms at least a part of an exterior surface of the drug dosage form. In some embodiments, the surface of the exterior lid and a portion of the first component form an exterior surface of the drug dosage form. In some embodiments, a portion of the exterior lid is operably connected to the dosage form such that the exterior lid hinges outwardly from the drug dosage form due to swelling of the swellable component. In some embodiments, the exterior lid is configured such that swelling of the swellable component moves the entire exterior lid outwardly from the drug dosage form. In some embodiments, the drug dosage form further comprises one or more swellable components each comprising an exterior lid.

In some embodiments, the swellable component and the first component are configured as two separate layers.

In some embodiments, the drug dosage form further comprises a second component, wherein the first component and the second component are connect, at least in part, via the swellable component. In some embodiments, the swelling of the swellable component shifts the relative positions of the first component and the second component, thereby increasing the dimension of the drug dosage form. In some embodiments, the first component has a first axis from a first proximal end to a first distal end, wherein the second component has a second axis from a second proximal end to a second distal end, and wherein the swellable component has a central axis, wherein upon swelling of the swellable component, the first axis and the second axis are shifted relative to each other. In some embodiments, the first axis and the second axis are aligned with each other without swelling of the swellable component, and rotate relative to each other upon swelling of the swellable component. In some embodiments, the first axis and the second axis become perpendicular to each other upon swelling of the swellable component. In some embodiments, at least a portion of the first component is configured to interface with the second component such that it creates a point of rotation.

In some embodiments, the first axis and the second axis are aligned with each other, and are shifted away from the central axis upon swelling of the swellable component. In some embodiments, the drug dosage form further comprises a first guiding track and a second guiding track, wherein the upon swelling of the swellable component the first component shifts along with the first guiding track away from the central axis, and wherein the second component shifts along with the second guiding track away from the central axis. In some embodiments, the drug dosage form further comprises a third component, a third guiding track, a fourth component, and a fourth guiding track, wherein upon swelling of the swellable component the third component shifts along with the third guiding track away from the central axis, and the fourth component shifts along with the fourth guiding track away from the central axis.

In some embodiments, the first component comprises a first drug. In some embodiments, the second component comprises a second drug. In some embodiments, the third component comprises a third drug. In some embodiments, the fourth component comprises a fourth drug.

In some embodiments, the swellable component comprises a core drug. In some embodiments, the swellable component does not comprise a drug.

In some embodiments, at least two of the first drug, the second drug, the third drug, the fourth drug, and the core drug are the same. In some embodiments, at least two of the first drug, the second drug, the third drug, the fourth drug, and the core drug are the different from each other.

In some embodiments, at least one of the first drug, the second drug, the third drug, the fourth drug, and the core drug is poorly water soluble.

In some embodiments, any one of the first drug, the second drug, the third drug, the fourth drug, and the core drug is in a compartment embedded in a substrate material.

In some embodiments, the first component, the second component, the third component, and/or the fourth component comprises a drug contained within a compartment. In some embodiments, the compartment comprises a plug.

In some embodiments, the first component, the second component, the third component, and/or the fourth component comprises more than one drug-filled compartment.

In some embodiments, the drug dosage form is an oral drug dosage form.

Provided herein, in other aspects, is a commercial batch of any drug dosage form described herein, wherein the commercial batch has a standard deviation of about 0.05 or less for each of the following: an amount of a drug in the drug dosage form; weight of the drug dosage form; a largest crossing dimension of the oral drug dosage form; a crossing dimension perpendicular to the largest crossing dimension of the oral drug dosage form; a largest crossing dimension of the oral drug dosage form after swelling of the swelling component; and a crossing dimension perpendicular to the largest crossing dimension of the oral drug dosage form after swelling of the swelling component. In some embodiments, the commercial batch comprises at least about 1000 of the drug dosage forms. In some embodiments, the drug dosage form is made by a 3D printing technique.

Provided herein, in other aspects, is a method of three-dimensional (3D) printing of any drug dosage form described herein, the method comprising: (a) dispensing the first component, or a portion thereof; and (b) dispensing the swellable component, or a portion thereof. In some embodiments, the dispensing is via melt extrusion deposition (MED). In some embodiments, dispensing of the first component, or a portion thereof, and dispensing of the swellable component, or a portion thereof, are performed by different printing heads.

Provided herein, in other aspects, is a method for preparing a drug dosage form by three-dimensional (3D) printing, wherein the drug dosage form comprises a first component, a second component, and a swellable component, the method comprising: (a) dispensing a material of the first component; (b) dispensing a material of the second component; and (c) dispensing a material of the swellable component. In some embodiments, the dispensing is via melt extrusion deposition (MED). In some embodiments, dispensing of each material is performed by a different printing head.

In some embodiments, the drug dosage form is 3D printed using a layer-by-layer technique.

Provided herein, in other aspects, is a method of delivering a drug in an individual and allowing the drug to be retained in the stomach of the individual for an extended time, the method comprising orally administering to the individual any drug dosage form described herein.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### BRIEF DESCRIPTION OF THE DRAWINGS

FIGS. 1A-1J show diagrams of exemplary drug dosage forms comprising a swellable component comprising an exterior lid (e.g., FIG. 1A, 104).

FIGS. 2A-2C show diagrams of exemplary drug dosage forms comprising a first component in the form of a layer (e.g., FIG. 2A, layer 202) and a swellable component in the form of a layer (e.g., FIG. 2A, layer 204).

FIGS. 3A-3C show diagrams of exemplary drug dosage forms comprising a first component (e.g., FIG. 3A, 302) having a first axis (e.g., FIG. 3A, 308) and a second component (e.g., FIG. 3B, 304) having a second axis (e.g., FIG. 3A, 310), wherein upon swelling of the swellable component (e.g., FIG. 3A, 306) the first axis and the second axis rotate relative to each other (FIG. 3B).

FIGS. 4A-4D show diagrams of exemplary drug dosage forms both prior to swelling (FIGS. 4A and 4C) and after swelling (FIGS. 4B and 4D) of a swellable component, wherein upon swelling of the swellable component a point on a first axis of a first component (e.g., FIG. 4A, 412) is shifted away from a central axis of the swellable component (e.g., FIG. 4A, 402).

### DETAILED DESCRIPTION

Provided herein, in some aspects, is a drug dosage form comprising a swellable component, wherein the swellable component, or at least a portion thereof, swells upon exposure to gastrointestinal fluid, and wherein the swelling of the swellable component increases the dimension of the drug dosage form, thereby allowing the drug to be retained in the stomach for an extended time (e.g., at least about 24 hours). The drug dosage forms described herein are based, at least in part, on the inventors' unique insights and findings regarding the design, construction, and manufacture of drug dosage forms comprising a swellable component that undergoes a precisely designed size change upon swelling of the swellable component such that the drug dosage form is retained in the stomach. The drug dosage forms of the present application can be designed to have controlled swelling (e.g., size, direction, shape, and speed) of the swellable component such that the expanded form of the drug dosage form is of a certain form that is mechanically sound to enable stomach retention for extended periods of time without interfering with normal stomach function. The drug dosage forms of the present application also can be configured to release a drug therefrom following any desired drug release profile (e.g., a sustained-release profile, a delayed-sustained release profile, a pulsed release profile). The inventors' have demonstrated that such drug dosage forms can be readily manufacturing using three-dimensional (3D) printing techniques.

Thus, in some aspects, provided is a drug dosage form comprising a first component and a swellable component, wherein the first component and the swellable component are connected, wherein at least one of the first component and the swellable component comprises a drug, wherein the swellable component, or a portion thereof, swells upon exposure to gastrointestinal fluid, and wherein the swelling of the swellable component, or the portion thereof, increases the dimension of the drug dosage form, thereby allowing the drug to be retained in the stomach for an extended prior of time, e.g., at least about 24 hours. In some embodiments, the first component surrounds, including partially surrounds, the swellable component, e.g., forms a shell around at least a portion of the swellable component.

In other aspects, provided is a drug dosage form comprising a first component and a swellable component, wherein the first component and the swellable component are connected, wherein the swellable component comprises an exterior lid and a swelling agent, wherein the exterior lid is connected to the swelling agent, wherein the exterior lid is configured such that it forms at least a part of an exterior surface of the drug dosage form, wherein at least one of the first component and the swellable component comprises a drug, wherein the swellable component, or a portion thereof, such as the swelling agent, swells upon exposure to gastrointestinal fluid, and wherein the swelling of the swellable component, or the portion thereof, increases the dimension of the drug dosage form, thereby allowing the drug to be retained in the stomach for at least about 8 hours, such as at least about any of 12 hours, 18 hours, or 24 hours.

In other aspects, provided is a drug dosage form comprising a first component and a swellable component, wherein the first component is a first layer, wherein the swellable component is a second layer, wherein the first component and the swellable component are connected, wherein at least one of the first component and the swellable component comprises a drug, wherein the swellable component swells upon exposure to gastrointestinal fluid, and wherein the swelling of the swellable component increases the dimension of the drug dosage form, thereby allowing the drug to be retained in the stomach for at least about 8 hours, such as at least about any of 12 hours, 18 hours, or 24 hours. In some embodiments, the swellable component does comprise an exterior lid.

In other aspects, provided is a drug dosage form comprising a first component, a second component, and a swellable component, wherein the first component and the second component are connect, at least in part, via the swellable component, wherein at least one of the first component, the second component, and the swellable component comprises a drug, wherein the first component has a first axis from a first proximal end to a first distal end, wherein the second component has a second axis from a second proximal end to a second distal end, wherein the swellable component has a central axis, wherein the first axis and the second axis are aligned, such as parallel or substantially parallel, with each other without swelling of the swellable component (such as in the administration state), and rotate relative to each other upon swelling of the swellable component, wherein the swellable component swells upon exposure to gastrointestinal fluid, and wherein the swelling of the swellable component, or the portion thereof, increases the dimension of the drug dosage form, thereby allowing the drug to be retained in the stomach for at least about 8 hours, such as at least about any of 12 hours, 18 hours, or 24 hours.

In other aspects, provided is a drug dosage form comprising a first component, a second component, and a swellable component, wherein the first component and the second component are connect, at least in part, via the swellable component, wherein at least one of the first component, the second component, and the swellable component comprises a drug, wherein the first component has a first axis from a first proximal end to a first distal end, wherein the second component has a second axis from a second proximal end to a second distal end, wherein the swellable component has a central axis, wherein the first axis and the second axis are aligned with each other (e.g., when the first axis and the second axis are position top to bottom through the drug dosage form), and are shifted away from the central axis upon swelling of the swellable component, wherein the swellable component swells upon exposure to gastrointestinal fluid, and wherein the swelling of the swellable component, or the portion thereof, increases the dimension of the drug dosage form, thereby allowing the drug to be retained in the stomach for at least about 8 hours, such as at least about any of 12 hours, 18 hours, or 24 hours. In some embodiments, the first axis, the second axis, and central axis are parallel or substantially parallel. For purposes of describing the drug dosage form, in some embodiments, a first axis and a second axis (as guided by the direction movement of the expanding dosage form) may each comprise a point, wherein a point on the first axis and a point on the second axis are shifted away from the central axis upon swelling of the swellable component. In some embodiments, the first axis may In some embodiments, the drug dosage form further comprises a first guiding track and a second guiding track, wherein the upon swelling of the swellable component the first component shifts along with the first guiding track away from the central axis, and wherein the second component shifts along with the second guiding track away from the central axis. In some embodiments, the drug dosage form further comprises a third component, a third guiding track, a fourth component, and a fourth guiding track, wherein upon swelling of the swellable component the third component shifts along with the third guiding track away from the central axis, and the fourth component shifts along with the fourth guiding track away from the central axis.

In other aspects, provided is a commercial batch of a drug dosage form described herein. In some embodiments, the commercial batch comprises at least about 1000 of the drug dosage forms. In some embodiments, the commercial batch has a standard deviation of about 0.05 or less for each of the following: an amount of a drug in the drug dosage form; weight of the drug dosage form; a largest crossing dimension of the oral drug dosage form; a crossing dimension perpendicular to the largest crossing dimension of the oral drug dosage form; a largest crossing dimension of the oral drug dosage form after swelling of the swelling component; and a crossing dimension perpendicular to the largest crossing dimension of the oral drug dosage form after swelling of the swelling component.

In other aspects, provided is a method of three-dimensional (3D) printing of a drug dosage form described herein. In some embodiments, wherein the drug dosage form comprises a first component and a swellable component, the method comprises: (a) dispensing the first component, or a portion thereof; and (b) dispensing the swellable component, or a portion thereof. In some embodiments, wherein the drug dosage form comprises a first component, a second component, and a swellable component, the method comprises: (a) dispensing a material of the first component; (b) dispensing a material of the second component; and (c) dispensing a material of the swellable component.

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the term “individual” refers to a mammal and includes, but is not limited to, human, bovine, horse, feline, canine, rodent, rat, mouse, dog, or primate. In some embodiments, the individual is a human individual.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### II. Drug Dosage Forms for Stomach Retention

The drug dosage forms provided herein comprise a swellable component configured to swell upon exposure to gastrointestinal fluid, wherein the swelling of the swellable component, or a portion thereof, leads to an increase in the dimension(s) of the drug dosage form such that the drug dosage form is retained in the stomach for an extended period of time. In some embodiments, the drug dosage form is configured in view of desired functions or attributes needed during the administration life cycle of the drug dosage form and characteristics of the individual to whom the drug dosage form is administered. For example, in some embodiments, the drug dosage form has an administration state, wherein the administration state of the drug dosage form is configured for oral administration to an individual, e.g., the drug dosage form is configured such that the drug dosage form can be swallowed and travel to the stomach. In some embodiments, the drug dosage form has a stomach retention state, wherein the stomach retention state of the drug dosage form is configured such that the dimension(s) of the drug dosage form prevent passage of drug dosage through the pylorus, or a portion thereof, thereby resulting in stomach retention. In some embodiments, the drug dosage form has an expulsion state, wherein the expulsion state of the drug dosage form, or components thereof, is such that passage through the pylorus is possible and the drug dosage form is cleared from the stomach. As detailed herein, numerous mechanisms are described that enable the swelling of the swellable component, or a portion thereof, to increase the dimension of the drug dosage form (e.g., transition from administration state to stomach retention state) such that the drug dosage form is retained in the stomach for an extended period of time.

### A. Exemplary Mechanisms of Drug Dosage Forms

In some embodiments, the drug dosage form comprises a first component and a swellable component, wherein the first component and the swellable component are connected, wherein the swellable component comprises an exterior lid and a swelling agent, wherein the exterior lid is connected to the swelling agent, wherein the exterior lid is configured such that it forms at least a part of an exterior surface of the drug dosage form, wherein at least one of the first component and the swellable component comprises a drug, wherein the swellable component, or a portion thereof, such as the swelling agent, swells upon exposure to gastrointestinal fluid, and wherein the swelling of the swellable component, or the portion thereof, increases the dimension of the drug dosage form, thereby allowing the drug to be retained in the stomach for an extended period of time. In some embodiments, the surface of the exterior lid and a portion of the first component form an exterior surface of the drug dosage form. In some embodiments, portion of the exterior lid is operably connected to the drug dosage form such that the exterior lid hinges outwardly from the drug dosage form due to swelling of the swellable component. In some embodiments, the exterior lid is configured such that swelling of the swellable component moves the entire exterior lid outwardly from the drug dosage form. The drug may be contained in any one or more of the described features of the drug dosage form.

In some embodiments, the drug dosage form comprises more than one, such as 2, 3, 4, or 5, swellable components. In some embodiments, each swellable component comprises an exterior lid and a swelling agent.

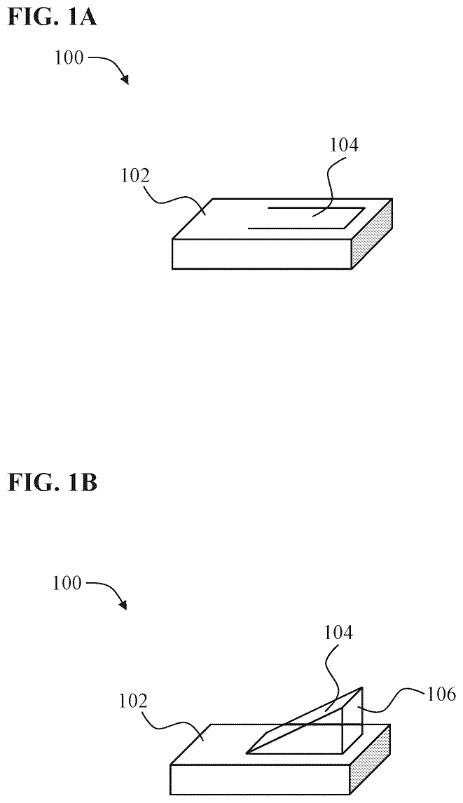

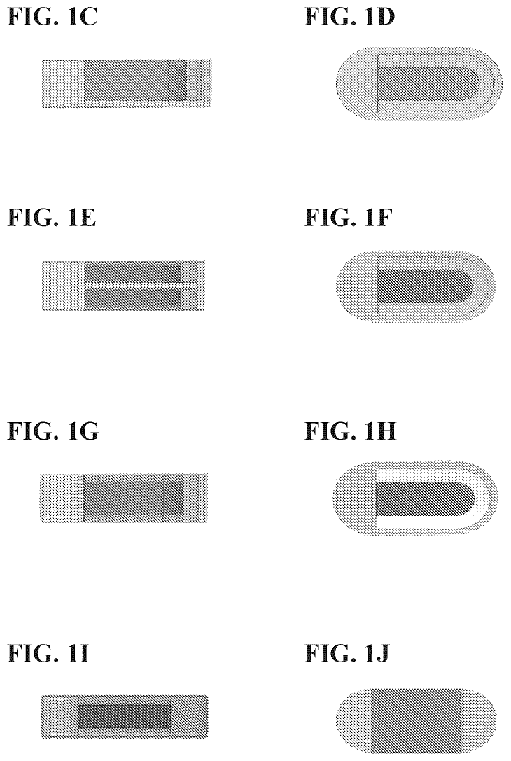

For purposes of illustration, as shown in FIG. 1A, the drug dosage form 100 comprises a first component 102 and a swellable component, wherein the swellable component comprises a swelling agent and an exterior lid 104. In FIG. 1A, prior to expansion of the swellable component, the swelling agent is in an internal space of the drug dosage form, and the exterior lid of the swellable component forms a surface of the exterior of the drug dosage form. In some embodiments, the drug dosage form is configured to allow gastrointestinal fluid to penetrate the drug dosage form and contact the swelling agent. In some embodiments, the drug dosage form comprises a feature, such as a pore, at, around, and/or within the exterior lid to allow gastrointestinal fluid to penetrate the drug dosage form and contact the swelling agent. In FIG. 1B, after expansion of the swelling agent 106 of the swellable component, the dimensions of the drug dosage form 100 are increased relative to the pre-expansion state (e.g., the administration state). In some embodiments, a portion of the exterior lid of the swellable component remains operably connected to the drug dosage form (e.g., a hinge mechanism demonstrated in FIG. 1B). In some embodiments, the exterior lid is completely separated from the original surface of the drug dosage form (e.g., a sliding-drawer mechanism; not illustrated). In some embodiments, the exterior lid provides structural and/or mechanical stability to the drug dosage form, or components thereof, such as the swelling agent. In some embodiments, the exterior lid is connected to the swelling agent such that the exterior lid and swelling agent remain connected during swelling of the swelling agent. In some embodiments, the drug dosage form 100 comprises a first component 102. In any embodiments described herein, the first component and/or the swellable component comprise a drug.

Additional exemplary drug dosage forms comprising a swellable component comprising a swelling agent and an exterior lid are provided in FIGS. 1C-1J. As shown in FIGS. 1C and 1D, the drug dosage form is in the shape of a capsule and comprises a single swellable component comprising a U-shaped exterior lid and a swelling agent, wherein the U-shaped exterior lid forms an exterior surface of the drug dosage form. As shown in FIGS. 1E and 1F, the drug dosage form is in the shape of a capsule and comprises two swellable components, each swellable component comprising a U-shaped exterior lid and a swelling agent, wherein the U-shaped exterior lid forms an exterior surface of the drug dosage form. As shown in FIGS. 1G and 1H, the drug dosage form is in the shape of a capsule and comprises a single swellable component comprising two U-shaped exterior lids and a swelling agent, wherein each U-shaped exterior lid forms an exterior surface of on opposing sides of the drug dosage form. In FIGS. 1C-1H, upon swelling of the swellable agent of the swelling component, the U-shaped exterior lid protrudes from the drug dosage form using a hinge mechanism As shown in FIGS. 1I and 1J, the drug dosage form is in the shape of a capsule and comprises a swellable component that upon swelling protrudes from the drug dosage form using a sliding-drawer mechanism.

In some embodiments, the drug dosage form comprises a first component and a swellable component, wherein the first component is a first layer, wherein the swellable component is a second layer, wherein the first component and the swellable component are connected, wherein at least one of the first component and the swellable component comprises a drug, wherein the swellable component swells upon exposure to gastrointestinal fluid, and wherein the swelling of the swellable component increases the dimension of the drug dosage form, thereby allowing the drug to be retained in the stomach for an extended period of time. In some embodiments, the drug dosage form does not comprise an exterior lid connected to the swellable component. In some embodiments, the swelling material of the swellable component forms at least one exterior surface of the drug dosage form prior to swelling of the swellable component (e.g., in an administration state). The drug may be contained in any one or more of the described features of the drug dosage form.

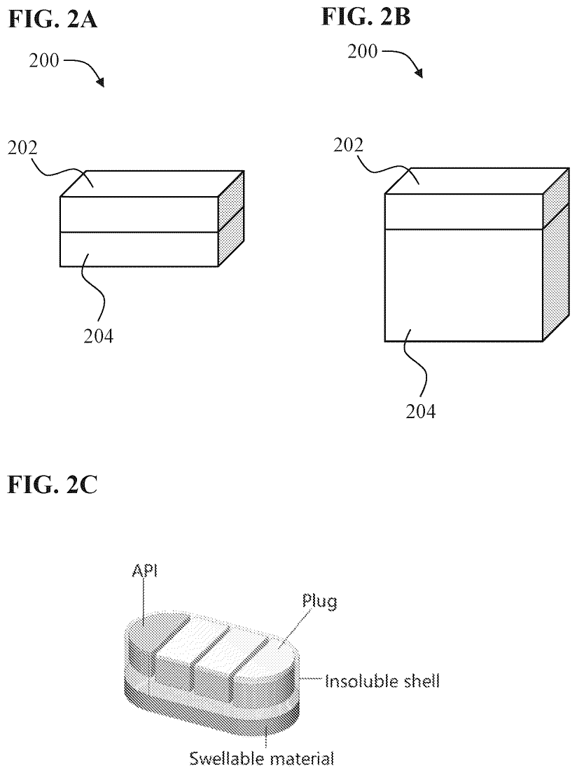

For purposes of illustration, as shown in FIG. 2A, the drug dosage form 200 comprises a first component layer 202 and a swellable component layer 204, wherein the first component layer 202 and the swellable component layer 204 are connected. FIG. 2A illustrated the drug dosage form 200 prior to expansion of the swellable component. In FIG. 2B, after expansion of the swellable component 204, the dimensions of the drug dosage form 200 are increased relative to a pre-expansion state (e.g., the administration state). In any embodiments described herein, the first component and/or the swellable component comprise a drug. For example, in some embodiments, the first component comprises a drug. As illustrated in FIG. 2C, the first component may comprises a plurality of compartment comprising a drug (API), wherein one or more of the compartments comprises a feature to control the release of the drug from the drug dosage form, such as a plug. As shown in FIG. 2C, the first component comprises an insoluble shell comprising the compartments, such that the drug is released from a specific aspect of the drug dosage form. In some embodiments, the one or more compartments are configures to release the drug from the drug dosage form in a pulsed manner, e.g., the compartments release drug therefrom in non-overlapping and/or overlapping series over time. In some embodiments, the drug dosage form comprise a gas-filled compartment, e.g., to provide buoyancy to the drug dosage form.

In some embodiments, the drug dosage form comprises a first component, a second component, and a swellable component, wherein the first component and the second component are connect, at least in part, via the swellable component, wherein at least one of the first component, the second component, and the swellable component comprises a drug, wherein the first component has a first axis from a first proximal end to a first distal end, wherein the second component has a second axis from a second proximal end to a second distal end, wherein the first axis and the second axis are aligned with each other (such as in a parallel or substantially parallel fashion) without swelling of the swellable component (such as in the administration state), and rotate relative to each other (such as out of the parallel or substantially parallel fashion) upon swelling of the swellable component, wherein the swellable component swells upon exposure to gastrointestinal fluid, and wherein the swelling of the swellable component, or the portion thereof, increases the dimension of the drug dosage form, thereby allowing the drug to be retained in the stomach for an extended period of time. In some embodiments, the first axis of the first component and the second axis of the second component become perpendicular to each other upon swelling of the swellable component. In some embodiments, the drug dosage form comprises a locking feature configured to fix the first component and second component in a position after swelling of the swellable component. In some embodiments, the first component and the second component are connected, at least in part. In some embodiments, the first component and the second component are connected, at least in part, via a feature, such as a protrusion or rod, of the first component and/or the second component. In some embodiments, at least a portion of the first component is configured to interface with the second component such that it creates a point of rotation. In some embodiments, the first component and the second component are connected, at least in part, via the swellable component. The drug may be contained in any one or more of the described features of the drug dosage form.

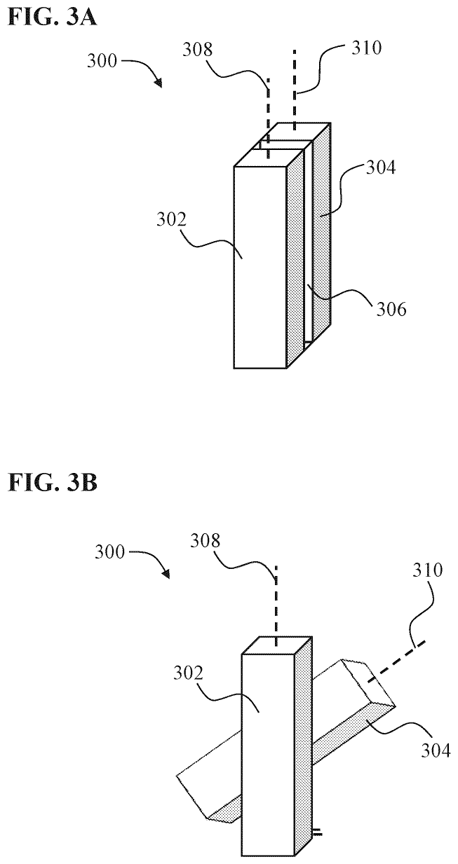

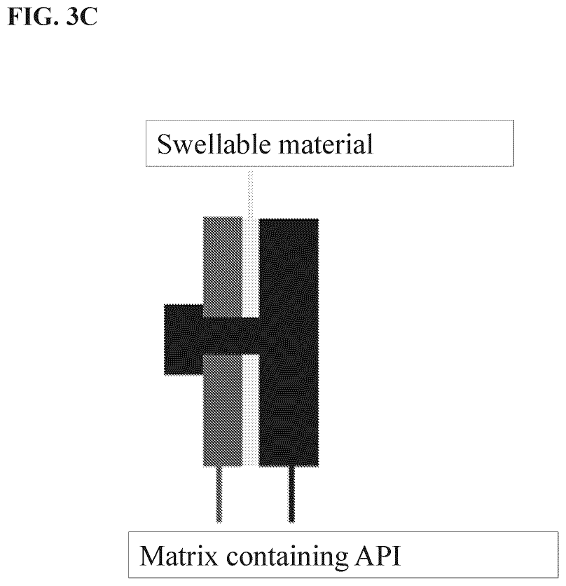

For purposes of illustration, as shown in FIG. 3A, the drug dosage form 300 comprises a first component 302, a second component 304, and a swellable component 306, wherein the first component 302 and the second component 304 are connect, at least in part, via the swellable component 306. The first component 302 in the drug dosage form 300 has a first axis 308 from a first proximal end to a first distal end, and the second component 304 has a second axis 310 from a second proximal end to a second distal end. As shown in FIG. 3A, without swelling of the swellable component (such as in the administration state), the first axis 308 and the second axis 310 are aligned with each other (such as in a parallel or substantially parallel fashion). As shown in FIG. 3B, upon swelling of the swellable component, the first axis 308 and the second axis 310 rotate relative to each other (such as out of the parallel or substantially parallel fashion) to increase a dimension of the drug dosage form. In some embodiments, at least one of the first component, the second component, and the swellable component comprises a drug.

In FIG. 3C, depicted is a cross-sectional diagram of an exemplary drug dosage form, similar to the drug dosage form in FIGS. 3A-3B, wherein the first component and the second component are connected, at least in part, via a rod extending from the first component. As shown in FIG. 3C, both the first component and the second component comprise a matrix containing drug (API).

In some embodiments, the drug dosage form comprises a first component, a second component, and a swellable component, wherein the first component and the second component are connected, at least in part, via the swellable component, wherein at least one of the first component, the second component, and the swellable component comprises a drug, wherein the first component has a first axis from a first proximal end to a first distal end, wherein the second component has a second axis from a second proximal end to a second distal end, wherein the swellable component has a central axis, wherein the first axis and the second axis are aligned with each other, and a point on the first axis and a point on second axis are shifted away from one another upon swelling of the swellable component, wherein the swellable component swells upon exposure to gastrointestinal fluid, and wherein the swelling of the swellable component, or the portion thereof, increases the dimension of the drug dosage form, thereby allowing the drug to be retained in the stomach for an extended period of time. In some embodiments, the swelling of the swellable component results in the movement of a first component relative to the other components of the drug dosage form. In some embodiments, the swelling of the swellable component results in the movement of a first component and a second component relative to the other components of the drug dosage form. In some embodiments, the point on the first axis is shifted away from a central axis of the swellable component, e.g., only the point on the first axis is shifted away from the central axis and not a point of a second axis of a second component. In some embodiments, the point on the first axis and the point on the second axis are shifted away from a central axis of the swellable component.

In some embodiments, the drug dosage form comprises a guiding track, wherein the drug dosage form is configured so that a component, such as a first component, slides on the guiding track. In some embodiments, the drug dosage form further comprises a first guiding track and a second guiding track, wherein the upon swelling of the swellable component the first component shifts along with the first guiding track away from the central axis, and wherein the second component shifts along with the second guiding track away from the central axis. Generally, such drug dosage forms may have any number of components in addition to the swellable component, such as a first component, second component, third component, fourth component, fifth component, sixth component, seventh component, and an eighth component. In some embodiments, one or more, including all, of the components have a guiding track. For example, in some embodiments, the drug dosage form further comprises a third component, a third guiding track, a fourth component, and a fourth guiding track, wherein upon swelling of the swellable component the third component shifts along with the third guiding track away from the central axis, and the fourth component shifts along with the fourth guiding track away from the central axis. The drug may be contained in any one or more of the described features of the drug dosage form.

In some embodiments, the guiding track is configured to prevent the component from leaving the drug dosage form. In some embodiments, the component comprises a feature that engages the guiding track. In some embodiments, the swelling of the swellable component pushes the component along the guiding track. In some embodiments, the drug dosage form is configured with a feature that stops and/or locks the component at a pre-determined position along the guiding track.

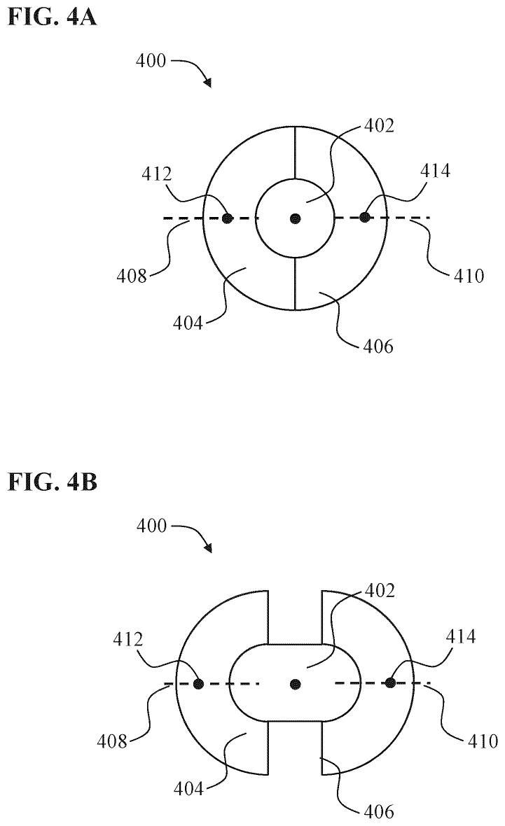

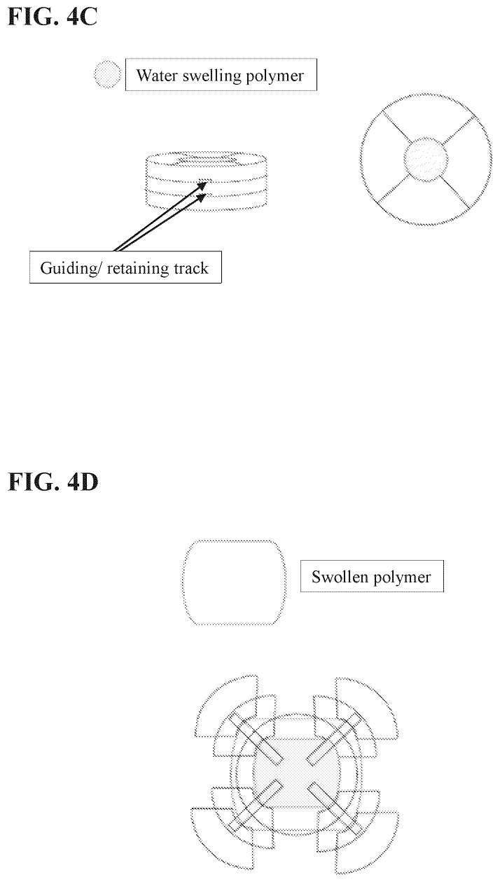

For purposes of illustration, as shown in FIG. 4A, the drug dosage form 400 comprises a first component 404, a second component 406, and a swellable component 402, wherein the first component 404 and the second component 406 are connected, at least in part, via the swellable component 402. The first component 404 has a first axis 408 from a first proximal end to a first distal end, the second component 406 has a second axis 410 from a second proximal end to a second distal end, and the swellable component 402 has a central axis. The first axis 408 and the second axis 410 are aligned with each other (e.g., on the same line, although the alignments encompassed in the present application does not need to be on the same line). A point 412 on the first axis 408, a point 414 on the second axis 410, and a central axis of the swellable component 402 are noted in FIGS. 4A and 4B to help track movement on the components during expansion of the swellable component 402. As shown in FIG. 4B, upon swelling of the swellable component 402, the point 412 on the first axis 408 and the point 414 on second axis 410 are shifted away from one another, and away from the central axis, noted by a point, of the swellable component 402. At least one of the first component, the second component, and the swellable component may comprises a drug.

As shown in FIGS. 4C and 4D, the drug dosage form may comprise a plurality of components and a swellable component. In FIG. 4C, the dosage form comprises layers of components and guiding tracks. As shown in FIG. 4D, upon expansion of the swellable component, the components of the drug dosage form expand in desired directions such that the overall dimensions of the drug dosage form are increased thereby allowing the drug to be retained in the stomach for an extended period of time.

### B. Characteristics of the Drug Dosage Forms in an Expanded State (e.g., the Stomach Retention State)

As described herein, the swelling of a swellable component, or a portion thereof, increases the dimension(s) of a drug dosage form, thereby allowing the drug to be retained in the stomach for an extended period of time. In some embodiments, the expanded state of a drug dosage form is referred to as a stomach retention state. One of ordinary skill in the art will readily appreciate that characteristics of the drug dosage form, such as size, during the swelling of the swellable component, or a portion thereof, may be dynamic and change over time. The description of certain states of the drug dosage form, such as stomach retention state, is not intended to limit the disclosure herein to only that one static embodiment of the drug dosage form.

In some embodiments, when the drug dosage form is in an expanded state (e.g., the stomach retention state), the drug dosage form is a size that inhibits and/or prevents passage of the drug dosage form through an aspect of the pylorus (such as the pyloric antrum, the pyloric canal, or the pyloric orifice create by the pyloric sphincter) to the duodenum. In some embodiments, when the drug dosage form is in an expanded state, the drug dosage form is a size that inhibits and/or prevents passage of the drug dosage form through the pyloric orifice created by the pyloric sphincter. In some embodiments, when the drug dosage form is in an expanded state, at least two perpendicular dimensions of the drug dosage form are each independently at least about 2 cm to about 7 cm in length, such as at least about 2 cm to about 5 cm in length, about 3 cm to about 6 cm in length, or about 4 cm to about 7 cm in length. In some embodiments, when the drug dosage form is in an expanded state, at least two perpendicular dimensions of the drug dosage form are each independently at least about 2 cm, such as at least about any of 2.5 cm, 3 cm, 3.5 cm, 4 cm, 4.5 cm, 5 cm, 5.5 cm, 6 cm, 6.5 cm, or 7 cm, in length. In some embodiments, when the drug dosage form is in an expanded state, at least two perpendicular dimensions of the drug dosage form are each independently about any of 2 cm, 2.5 cm, 3 cm, 3.5 cm, 4 cm, 4.5 cm, 5 cm, 5.5 cm, 6 cm, 6.5 cm, or 7 cm, in length. In some embodiments, when the drug dosage form is in an expanded state, one dimension of the at least two perpendicular dimensions is different from another dimension. In some embodiments, when the drug dosage form is in an expanded state, one dimension of the at least two perpendicular dimensions is the same as another dimension.

The drug dosage forms described herein are configured to be retained in the stomach for an extended period of time, such as compared to a drug dosage form without a gastric retention feature. In some embodiments, the drug dosage form is configured to be retained in the stomach for about 8 hours to about 7 days, such as any of about 8 hours to about 24 hours, about 18 hours to about 30 hours, about 20 hours to about 28 hours, about 1 day to about 3 days, or about 3 days to about 7 days. In some embodiments, the drug dosage form is configured to be retained in the stomach for at least about 8 hours, such as at least about any of 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, or 36 hours. In some embodiments, the drug dosage form is configured to be retained in the stomach for at least about 1 day, such as at least about any of 2 days, 3 days, 4 days, 5 days, 6 days, or 7 days. In some embodiments, the drug dosage form is configured to be retained in the stomach for no longer than about 7 days, such as no longer than about any of 6 days, 5 days, 4 days, 3 days, 2 days, 36 hours, 30 hours, 24 hours, 18 hours, or 12 hours. In some embodiments, the drug dosage form is configured to be retained in the stomach for at about any of 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 2 days, 3 days, 4 days, 5 days, 6 days, or 7 days.

In some embodiments, the drug dosage form is configured such that the drug dosage form, or parts thereof, can pass through the pylorus and be expelled from the stomach. For example, in some embodiments, the first component and the second component disengage, such as break apart, and the components of the drug dosage form may then be expelled from the stomach. In some embodiments, one or more components of the drug dosage form, such as a first component, a second component, and/or a swellable component, erodes or dissolves, in whole or in part, such that the drug dosage form may then be expelled from the stomach. In some embodiments, the erosion or dissolution of a component, or a part thereof, of a drug dosage form is due to prolonged exposure to gastrointestinal fluids in the stomach (e.g., due to prolonged exposure to a low pH).

### C. Characteristics of the Drug Dosage Form in the Administration State

The oral drug dosage forms described herein may be formed in any number of shapes, sizes, weights, and appearances. As described herein, the drug dosage forms of the present application may take forms having different features (such as a size and shape) during the life cycle of the administered drug dosage form (e.g., the administration state and the stomach retention state). Unless otherwise stated, certain features of the administration state of the drug dosage forms disclosed herein are described below in this section.

In some embodiments, the drug dosage form is an oral drug dosage form. In some embodiments, the oral drug dosage form described herein is suitable for oral administration to a human individual. Such drug dosage forms of the present application can be, for example, any size, shape, or weight that is suitable for oral administration to specific human individuals, such as children and adults. In some embodiments, the drug dosage form is suitable for oral administration to an individual, wherein selection of size, shape, or weight of the drug dosage form is based on an attribute of the individual, e.g., one or more of height, weight, age, or a size of an anatomical feature.

In some embodiments, the surface, such as an exterior surface, of the drug dosage form has the shape of a capsule, circle, oval, bullet shape, arrow head shape, triangle, arced triangle, square, arced square, rectangle, arced rectangle, diamond, pentagon, hexagon, octagon, half moon, almond, or a combination thereof.

In some embodiments, the drug dosage form has a largest crossing dimension of about 5 mm to about 20 mm, such as any of about 5 mm to about 15 mm, about 6 mm to about 13 mm, or about 7 to about 11 mm. In some embodiments, the drug dosage form has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the drug dosage form has a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the drug dosage form has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the largest crossing dimension is measured across a surface, such as an exterior surface, of the drug dosage form. In some embodiments, the largest crossing dimension is measured crossing through a drug dosage form.

In some embodiments, the drug dosage form has a crossing dimension perpendicular to the largest crossing dimension of about 5 mm to about 20 mm, such as any of about 5 mm to about 15 mm, about 6 mm to about 13 mm, or about 7 to about 11 mm. In some embodiments, the drug dosage form has a crossing dimension perpendicular to the largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the drug dosage form has a crossing dimension perpendicular to the largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the drug dosage form has a crossing dimension perpendicular to the largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the crossing dimension perpendicular to a largest crossing dimension is measured across a surface of the delayed sustained-release oral drug dosage form. In some embodiments, the crossing dimension perpendicular to a largest crossing dimension is measured crossing through a drug dosage form.

In some embodiments, the drug dosage form has a thickness of about 5 mm to about 20 mm, such as any of about 5 mm to about 15 mm, about 6 mm to about 13 mm, or about 7 to about 11 mm. In some embodiments, the drug dosage form has a thickness of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the drug dosage form has a thickness of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the drug dosage form has a thickness of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, the drug dosage form has a total weight of about 50 mg to about 1,000 mg, such as any of about 50 mg to about 100 mg, about 100 to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, or about 900 mg to about 1,000 mg. In some embodiments, the drug dosage form has a total weight of at least about 50 mg, such as at least about any of 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1,000 mg. In some embodiments, the drug dosage form has a total weight of less than about 1,000 mg, such as less than about an of 950 mg, 900 mg, 850 mg, 800 mg, 750 mg, 700 mg, 650 mg, 600 mg, 550 mg, 500 mg, 475 mg, 450 mg, 425 mg, 400 mg, 375 mg, 350 mg, 325 mg, 300 mg, 275 mg, 250 mg, 225 mg, 200 mg, 175 mg, 150 mg, 125 mg, 100 mg, 75 mg, or 50 mg. In some embodiments, the drug dosage form has a total weight of about any of 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1,000 mg.

### D. Integration of Drugs in the Drug Dosage Forms and Drug Release Profiles

The drug dosage forms described herein comprise one or more drugs. The drugs of the drug dosage forms can be released at any point of the life cycle of the drug dosage form. For example, in some embodiments, the drug dosage form is configured and formulated to release a drug when in an expanded state (e.g., a stomach retention state). In some embodiments, substantially all, such as at least about any of 70%, 75%, 80%, 85%, 90%, 95%, or 100, of the drug in the drug dosage form is release during the expanded state (such as the stomach retention state). In some embodiments, the drug dosage form is configured to release a drug prior to swelling of the swellable component. In some embodiments, the drug dosage form is configured to release a drug after leaving the stomach.

In some embodiments, the drug is a poorly-water soluble drug. In some embodiments, the poorly-water soluble drug is a Biopharmaceutics Classification System (BCS) Class II active pharmaceutical ingredient (API), e.g., a drug having high permeability and low solubility. In some embodiments, the drug is selected from the group consisting of aceclofenac, bicalutamide, carbamazepine, carvedilol, clotrimazole, cinnarizine, danazol, dapsone, estradiol, ezetimibe glibenclamide, fenofibrate, griseofulvin, ibuprofen, itraconazole, ketoconazole, mefenamic acid, naproxen, nevirapine, nifedipine, nitrofurantoin, nomegestrol acetate, phenytoin sodium salt, piroxicam, praziquantel, rifampicin, sulfamethoxazole, trimethoprim, and verapamil hydrochloride.

The drug dosage forms may be configured to release a drug based on any desired release profile. Generally, as the drug dosage forms described herein are designed to be retained in the stomach for an extended period of time, the release profile of at least one drug will be configured based on the expected stomach residency of the drug dosage form. In some embodiments, when two or more drugs are in the drug dosage form, the drug dosage form is configured to release each drug according to a desired release profile. In some embodiments, the drug dosage form is configured such that all drug content (or substantially all, such as at least about 90%) in the drug dosage form is released during the expected stomach residency of the drug dosage form. In some embodiments, the drug dosage form is configured such that an amount of the drug content (such as a second drug) in the drug dosage form is released after the drug dosage form, or components thereof, are expected to be expelled from the stomach. In some embodiments, the drug dosage form is formulated and configured such that the drug is released according to a delayed release profile, a sustained release profile, a delayed-sustained release profile, a zero-order release profile, a first-order release profile, an immediate release profile plus a sustained release profile, an immediate release profile plus a delayed release profile, an immediate release profile plus a delayed-sustained release profile, a pulsed release profile, an iterative pulsed release profile, an immediate release profile plus a pulsed release profile, or a combination thereof. For example, in some embodiments, the delayed release profile comprises release of the drug after at least about 2 hours following administration of the drug dosage form to the individual. In some embodiments, the sustained release profile comprises release of the drug for greater than a period of about 2 hours).

The drug dosage forms described herein may be configured and formulated to release a drug according to a desired drug release profile using a variety of techniques. In some embodiments, the release of the drug from the drug dosage form is based on the erosion of a drug-containing material, such as when the drug-containing material is exposed to gastrointestinal fluid. In some embodiments, the drug-containing material is configured as a layer having a pre-determined surface area, such a surface area exposed to the gastrointestinal fluid, thickness, and drug mass fraction, wherein these characteristics of the drug-containing material provide a desired drug release. In some embodiments, the drug-containing material is in the form of a multi-layered structure. In some embodiments, the drug-containing material is embedded in a material of a component, such as a first component or a swellable component, of the drug dosage form. Design, configurations, and materials of such drug-contain materials to provide a desired drug release are known in the art, e.g., see U.S. Pat. No. 10,350,822, which is hereby incorporated herein in its entirety.

In some embodiments, the drug dosage form is configured with a drug-containing compartment, wherein the compartment has an orifice from which drug is release from the drug dosage form. In some embodiments, the orifice is blocked with an erodible material, such as a plug. In some embodiments, the feature blocking the orifice, such that a drug is held within a compartment of a drug dosage form, is configured to no longer block the orifice at a desired time. For example, in some embodiments, the drug-containing compartment is sealed with an erodible plug, wherein the erodible plug dissolves at a certain time after administration to the individual to thereby release the drug from the drug dosage form. The timing of release can be based on, e.g., thickness of the plug and/or material of the plug. The drug-containing compartment can be configured in any component, such as a first component or a swellable component, of the drug dosage form. In some embodiments, the drug dosage form comprises a plurality of drug-containing compartments. In some embodiments, the component is a non-erodible material, such as an insoluble shell material. In some embodiments, the component erodes after the drug has left the drug-containing compartment, e.g., after the drug dosage form exits the stomach.

In some embodiments, the drug dosage form is configured such that a drug will leach from a material thereof.

The drug or drugs of the drug dosage forms described herein may be a part of any component described herein.

In some embodiments, wherein the drug dosage form comprises a first component comprising a drug. In some embodiments, wherein the drug dosage form comprises a second component comprising a drug. In some embodiments, wherein the drug dosage form comprises a third component comprising a drug. In some embodiments, wherein the drug dosage form comprises a fourth component comprising a drug. In some embodiments, wherein the drug dosage form comprises a swellable component comprising a drug. In some embodiments, the swelling agent of the swellable component comprises a drug. In some embodiments, wherein the drug dosage form comprises a plurality of components comprising a drug, each component comprises the same drug and/or two or more components comprise the same drug. In some embodiments, wherein the drug dosage form comprises a plurality of components comprising a drug, each component comprises a different drug and/or two or more components comprise a different drug. In some embodiments, the swellable component does not comprise a drug.

### B. Materials of Components of the Drug Dosage Forms

The materials used to form components of the drug dosage form, such as the first component and a swellable component, may be selected based on desired properties and/or functions thereof.

In some embodiments, the swellable component, or a portion thereof such as the swelling agent, is configured and formulated such that upon exposure to gastrointestinal fluid the swellable component swells to a desired size and/or shape. In some embodiments, the swellable component forms a pre-determined shape when swollen. In some embodiments, the shape of the swellable component after swelling is different than the shape of the swellable component prior to swelling. In some embodiments, the shape of the swellable component after swelling is the same shape of the swellable component prior to swelling. In some embodiments, the swellable component comprises a coating. In some embodiments, the coating of the swellable component delays the swelling of the swellable component, such as by inhibiting contact with gastrointestinal fluid and/or restraining swelling, for at least a pre-determined amount of time after administration of the drug dosage form to an individual.

In some embodiments, the swellable component, and material thereof, is configured to expand at a desired rate and/or with a desired force. For example, in some embodiments, the swellable component, and material thereof, is configured to quickly expand upon contact with gastrointestinal to prevent the drug dosage form from passing through the stomach prior to remaining in the stomach for a desired stomach residency period. In some embodiments, the swellable component, and material thereof, is configured to expand to a stomach retention state within about 5 minutes of contacting gastrointestinal fluid, such as within about any of 4.5 minutes, 4 minutes, 3.5 minutes, 3 minutes, 2.5 minutes, 2 minutes, 1.5 minutes, 1 minutes, or 30 seconds. In some embodiments, the swellable component, and material thereof, is configured to expand with the force necessary to move components of the drug dosage form to a stomach retention state.

In some embodiments, the swellable component comprises a gas-generating, such as an effervescent agent. In some embodiments, the gas-generating agent comprises a source of carbon dioxide. In some embodiments, the gas-generating agent comprises a carbonate salt, or a bicarbonate salt, or a combination thereof. Exemplary carbonate salts include, but are not limited to, sodium carbonate, sodium bicarbonate, potassium carbonate, potassium bicarbonate, calcium carbonate, calcium bicarbonate, magnesium carbonate, and magnesium bicarbonate. In some embodiment, the gas-generating agent is sodium bicarbonate. In some embodiments, the swellable component further comprises an acid or acid source. Exemplary acids or acid sources include, but are not limited to, citric acid or salts thereof, tartaric acid or salts thereof, fumaric acid or salts thereof, adipic acid or salts thereof, malic acid or salts thereof, and formic acid, sorbic acid, succinic acid or salts thereof, glacial acetic acid, salicylic acid, propionic acid, phosphoric acid or salts thereof, lactic acid, benzoic acid. In some embodiment, the source of acid is sodium formate. In some embodiment, the swellable component comprises sodium bicarbonate and sodium formate.

In some embodiments, the swellable component, such as the swellable agent of the swellable component, comprises a material selected from the group consisting of: a cross-linked product, and a shape memory material. In some embodiments, the swellable component, such as the swellable agent of the swellable component, comprises a material selected from the group consisting of: polyethylene oxide-polyethylene glycol (PEO-PEG) cross-linked polymer, polycaprolactone-polyethylene glycol-polycaprolactone (PCL-PEG-PCL), hydroxypropylcellulose, polyethylene oxide (PEO), such as high molecular weight PEO, sodium alginate, carbomer, high molecule weight hydroxypropyl cellulose (HPC), high molecular weight hydroxylpropyl methylcellulose or Hypromellose (HPMC), methyl cellulose (MC), high molecular polyvinyl alcohol (PVA), polyvinyl acetate (PVAc) and polyvinylpyrrolidone (PVP) 80/20, methacrylic ester copolymer, Poly [Butyl methacrylate, (2-dimethylaminoethyl) methacrylate, methyl methacrylate] with the ratio 1:2:1 (e.g. EUDRAGIT® E 100, EUDRAGIT® E 12,5, EUDRAGIT® E PO), Poly [Ethyl acrylate, methyl methacrylate] with the ratio 2:1 (e.g. EUDRAGIT® NE 30 D, EUDRAGIT® NE 40 D, EUDRAGIT® NM 30 D), Poly [methacrylic acid, methyl methacrylate] with the ratio 1:1 (e.g. EUDRAGIT® L 100, EUDRAGIT® L 12,5, EUDRAGIT® L 12,5 P), Poly [methacrylic acid, ethyl acrylate] with the ratio 1:1 (e.g. Acryl-EZE, Acryl-EZE 93A, Acryl-EZE MP, EUDRAGIT® L 30 D-55, EUDRAGIT® L 100-55, Eastacryl® 30 D, Kollicoat® MAE 30 DP, Kollicoat® MAE 100 P), Poly [methacrylic acid, methyl methacrylate] with the ratio 1:2 (e.g. EUDRAGIT® S 100, EUDRAGIT® S 12,5, EUDRAGIT® 12,5 P, Poly [Methyl acrylate, methyl methacrylate, methacrylic acid] with the ratio 7:3:1) (e.g. EUDRAGIT® FS 30 D), Poly [Ethyl acrylate, methyl methacrylate, trimethylammonioethyl methacrylate chloride] with the ratio 1:2:0.2 (e.g. EUDRAGIT® RL 100, EUDRAGIT® RLPO, EUDRAGIT® RL 30 D, EUDRAGIT® RL 12,5), Poly [Ethyl acrylate, methyl methacrylate, trimethylammonioethyl methacrylate chloride] with the ratio 1:2:0.1 (e.g. EUDRAGIT® RS 100, EUDRAGIT® RS PO, EUDRAGIT® RS 30 D, EUDRAGIT® RS 12,5), hydroxypropyl methylcellulose acetate succinate or hypromellose acetate succinate (HPMCAS), hydroxypropyl methylcellulose phthalate (HPMCP), or combinations thereof. In some embodiments, the shape memory material is selected from the group consisting of polyurethanes, block copolymer of polyethylene terephthalate (PET) and polyethyleneoxide (PEO), block copolymers containing polystyrene and poly(1,4-butadiene), an ABA triblock copolymer made from poly(2-methyl-2-oxazoline) and polytetrahydrofuran, polynorbornene (Norsorex, developed by CdF Chemie/Nippon Zeon), polynorbornene with partially substituted polyhedral oligosilsesquioxane (POSS), copolymer consisting of polycyclooctene (PCOE) and poly(5-norbornene-exo,exo-2,3-dicarboxylic anhydride) (PNBEDCA), poly(ester-urethane)s, composites composed of a polyol (soft segment) and a diisocyanate coupled with a chain extender (hard phase)(poly(ε-caprolactone) (PCL), poly(ethylene adipate) (PEA) glycol)), the combination of poly(ester-urethane)s (PURs) and PCL, ethylene oxide-ethylene terephthalate segmented copolymers, oligo(ε-caprolactone)- and oligo(p-dioxanone)-based PUR, poly(p-dioxanone)-b-poly(tetramethylene oxide glycol) multiblocky copolymers, poly(methyl methacrylate)-poly(ethylene glycol) (PMMA-PEG) semi-interpenetrated network (IPN), poly(cyclohexylmethacrylate) (PCHMA) main-chain cross-linked with difunctional PCL polymers, polymers with grafted short PEG side chains on the PCL main chains, Nafion®, copoly(ester-urethane) network, covalently cross-linked poly[ethylene-co-(vinyl acetate)] (cPEVA), combination of PCL and poly(tetramethylene ether)glycol (PTMEG), or combinations thereof. In some embodiments, the material may have a plurality of properties, including a swellable material and a shape memory material.

In some embodiments, the component, such as the first component, comprises an insoluble material, a pH-sensitive erosion material, e.g., a material that does not erode in the pH of the stomach, a slowly eroding material, e.g., a material that will erode after the drug dosage form, or components thereof, leave the stomach, or a combination thereof. In some embodiments, the component, such as the first component, comprises a material selected from the group consisting of: Poly [Butyl methacrylate, (2-dimethylaminoethyl) methacrylate, methyl methacrylate] with the ratio 1:2:1 (e.g. EUDRAGIT® E 100, EUDRAGIT® E 12,5, EUDRAGIT® E PO), Poly [Ethyl acrylate, methyl methacrylate] with the ratio 2:1 (e.g. EUDRAGIT® NE 30 D, EUDRAGIT® NE 40 D, EUDRAGIT® NM 30 D), Poly [methacrylic acid, methyl methacrylate] with the ratio 1:1 (e.g. EUDRAGIT® L 100, EUDRAGIT® L 12,5, EUDRAGIT® L 12,5 P), Poly [methacrylic acid, ethyl acrylate] with the ratio 1:1 (e.g. Acryl-EZE, Acryl-EZE 93A, Acryl-EZE MP, EUDRAGIT® L 30 D-55, EUDRAGIT® L 100-55, Eastacryl® 30 D, Kollicoat® MAE 30 DP, Kollicoat® MAE 100 P), Poly [methacrylic acid, methyl methacrylate] with the ratio 1:2 (e.g. EUDRAGIT® S 100, EUDRAGIT® S 12,5, EUDRAGIT® 12,5 P, Poly [Methyl acrylate, methyl methacrylate, methacrylic acid] with the ratio 7:3:1) (e.g. EUDRAGIT® FS 30 D), Poly [Ethyl acrylate, methyl methacrylate, trimethylammonioethyl methacrylate chloride] with the ratio 1:2:0.2 (e.g. EUDRAGIT® RL 100, EUDRAGIT® RLPO, EUDRAGIT® RL 30 D, EUDRAGIT® RL 12,5), Poly [Ethyl acrylate, methyl methacrylate, trimethylammonioethyl methacrylate chloride] with the ratio 1:2:0.1 (e.g. EUDRAGIT® RS 100, EUDRAGIT® RS PO, EUDRAGIT® RS 30 D, EUDRAGIT® RS 12,5), stearic acid, ethyl cellulose (EC), titanium dioxide, cellulose acetate phthalate (CAP), poly(lactide-co-glycolide) (PLGA), ethylene-vinyl acetate copolymer, polyethylene (PE), polycaprolactone (PCL), polylactic acid (PLA), cellulose acetate butyrate (CAB), cellulose acetate (CA), polyvinyl acetate (PVAc), polyvinyl acetal diethyl amino lactate (AEA), or a combination thereof.

In some embodiments, the material of the drug dosage form is a thermoplastic material. In some embodiments, the thermoplastic material is a thermoplastic polymer. In some embodiments, the thermoplastic material comprises any one or more of a plasticizer, and another additive, e.g., a filler, a binder, a lubricant, a glidant, and a disintegrant. In some embodiments, the additive is selected from the group consisting of a clay, a SiC nanoparticle, a Ni powder, a carbon nanotube, a carbon fiber, a carbon black, a graphene, a metal oxide (e.g., Fe3O4, TiO₂, ZnO), a silver (Ag) nanoparticle, a gold (Au) nanoparticle, a silver and gold nanoparticle, a nanorod, a nanowhisker, a nanowire, and a cellulose nanocrystals.

### E. Additional Features of the Drug Dosage Forms

In some embodiments, the drug dosage form comprise a gas-filled compartment, e.g., to provide buoyancy to the drug dosage form. In some embodiments, the gas-filled compartment is embedded in a component, such as a first component or a swellable component. In some embodiments, the gas-filled compartment has an erodible plug, wherein the erodible plug is configured to open the gas-filled compartment at a point after administration of the drug dosage form to the individual.

In some embodiments, the drug dosage form comprises an additional feature, such as an outer coating, an outer layer, or an outer marking. In some embodiments, the outer coating or layer is a flavor coating. In some embodiments, the outer coating or layer is a sugar coating. In some embodiments, the outer coating or layer is a cosmetic coating. In some embodiments, the outer coating or layer is a color coating. In some embodiments, the outer coating or layer is a film coating. In some embodiments, the outer coating or layer is a polymer coating. In some embodiments, the outer coating completely surrounds the drug dosage form. In some embodiments, the outer layer forms a portion of the exterior of the drug dosage form. In some embodiments, the additional component is a label, such as a drug logo, company name or abbreviation, graphic, medication label, drug chemical name or abbreviation, drug specification, an identification barcode, or a combination thereof.

### III. Commercial Batches

In some aspects, provided herein is a commercial batch of the drug dosage forms described herein. In some embodiments, the commercial batch comprises at least about any of 100, 150, 200, 250, 500, 750, 1,000, 2,500, 5,000, 7,500, 10,000, 20,000, 30,000, 40,000, 50,000, 60,000, 80,000, 90,000 or 100,000 delayed sustained-release oral drug dosage forms described herein. In some embodiments, each of the drug dosage forms of the commercial batch are produced using the same technique, such as via three-dimensional (3D) printing.

In some embodiments, the commercial batch has a standard deviation of about 0.1 or less, such as 0.05 or less, for one or more of the following: an amount of a drug in the drug dosage form; weight of the drug dosage form; dimensions of the drug dosage form (such as in the administration state and/or the retention state); and time of stomach retention of the drug dosage form. In some embodiments, the dimension of the drug dosage form is a largest crossing dimension of the oral drug dosage form prior to swelling of the swelling component (such as in the administration state). In some embodiments, the dimension of the drug dosage form is a crossing dimension perpendicular to the largest crossing dimension of the oral drug dosage form prior to swelling of the swelling component (such as in the administration state). In some embodiments, the dimension of the drug dosage form is a largest crossing dimension of the oral drug dosage form after swelling of the swelling component (such as in a stomach retention state). In some embodiments, the dimension of the drug dosage form is a crossing dimension perpendicular to the largest crossing dimension of the oral drug dosage form after swelling of the swelling component (such as in a stomach retention state).

### IV. Methods of Making

In some aspects, provided herein are methods of making a drug dosage form described herein. In some embodiments, the method of making comprises a three-dimensional (3D) printing technique to form at least one of the components, or a portion thereof, of the drug dosage forms described herein.

As used herein, “printing,” “three-dimensional printing,” “3D printing,” “additive manufacturing,” or equivalents thereof, refers to a process that produces three-dimensional objects, such as delayed sustained-release oral drug dosage forms, layer-by-layer using digital designs. The basic process of three-dimensional printing has been described in U.S. Pat. Nos. 5,204,055; 5,340,656; 5,387,380; 5,503,785; and 5,633,021. Additional U.S. patents and patent applications that related to three-dimensional printing include: U.S. Pat. Nos. 5,490,962; 5,869,170; 6,530,958; 6,280,771; 6,514,518; 6,471,992; 8,828,411; U.S. Publication Nos. 2002/0015728; 2002/0106412; 2003/0143268; 2003/0198677; 2004/0005360. The contents of the above U.S. patents and patent applications are hereby incorporated herein by reference in their entirety. In some embodiments, an additive manufacturing technique is used to produce the drug dosage forms described herein. In some embodiments, a layer-by-layer technique is used to produce the drug dosage forms described herein. For example, in some embodiments, the layer-by-layer technique comprises dispensing one or more materials of an entire first layer of a drug dosage form, and then proceeding to dispense one or more materials of an entire second layer of the drug dosage form. In some embodiments, the layer, such as the first layer or second layer, is a cross-section of a drug dosage form. In some embodiments, the layer, such as the first layer or second layer comprises a portion of a first component and a portion of a swellable component of a drug dosage form. Because 3D printing may handle a range of pharmaceutical materials and control both composition and architecture locally, 3D printing is well suited to the fabrication of drug dosage forms with complex geometry and compositions in accordance with the present invention.

In some embodiments, layer, when used in reference to a components of the drug dosage form, e.g., a swellable component, refers to the configuration of a component of the drug dosage form and may comprise a plurality of printed layers of the same material. In some embodiments, the layer has a pre-determined fill density, such a three-dimensional printed fill density. In some embodiments, the layer comprises a plurality of printed layers between about 5 printed layers to about 2500 printed layers, such as between any of about 10 printed layers to about 2500 printed layers, about 25 printed layers to about 100 printed layers, about 50 printed layers to about 200 printed layers, about 100 printed layers to about 200 printed layers, about 150 printed layers to about 250 printed layers, about 200 printed layers to about 250 printed layers, about 500 printed layers to about 1000 printed layers, or about 2000 printed layers to about 2400 printed layers. In some embodiments, the thickness of a printed layer is no more than about 5 mm, such as no more than about any of 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm. In some embodiments, the thickness of a printed layer is about any of 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm.

Different 3D printing methods have been developed for manufacturing in terms of raw materials, equipment, and solidification. These 3D printing methods include binder deposition (see Gibson et al., Additive Manufacturing Technologies: 3D Printing, Rapid Prototyping, and Direct Digital Manufacturing, 2 ed. Springer, New York, 2015; Katstra et al., Oral dosage forms fabricated by three dimensional printing, J Control Release, 66, 2000; Katstra et al., Fabrication of complex oral delivery forms by three dimensional printing, Dissertation in Materials Science and Engineering, Massachusetts Institute of Technology, 2001; Lipson et al., Fabricated: The New World of 3D printing, John Wiley & Sons, Inc., 2013; Jonathan, Karim 3D printing in pharmaceutics: a new tool for designing customized drug delivery systems, Int J Pharm, 499, 2016), material jetting (see Jonathan, Karim, 3D printing in pharmaceutics: a new tool for designing customized drug delivery systems, Int J Pharm, 499, 2016), extrusion (see Gibson et al., Additive Manufacturing Technologies: 3D Printing, Rapid Prototyping, and Direct Digital Manufacturing. 2 ed. Springer, New York, 2015), and photopolymerization (see Melchels et al., A review on stereolithography and its application in biomedical engineering. Biomaterials, 31, 2010).

In some embodiments, the drug dosage forms described herein are 3D printed using an extrusion method. In some embodiments, the method of 3D printing comprises using a double screw extrusion method. In an extrusion process, material is extruded from robotically-actuated printing heads through printing nozzles. Unlike binder deposition, which requires a powder bed, extrusion methods can print on any substrate. A variety of materials can be extruded for three-dimensional printing, including thermoplastic materials disclosed herein, pastes and colloidal suspensions, silicones, and other semisolids. One extrusion printing method is melt extrusion deposition (MED), which used extruded material from a printing head to print layers of material to form the components of the drug dosage form. Another common type of extrusion printing is fused deposition modeling, which uses solid polymeric filaments for printing. In fused deposition modeling, a gear system drives the filament into a heated nozzle assembly for extrusion (see Gibson et al., Additive Manufacturing Technologies: 3D Printing, Rapid Prototyping, and Direct Digital Manufacturing, 2 ed. Springer, New York, 2015).

In some embodiments, the 3D printing is carried out by melt extrusion deposition (MED). In some embodiments, the melt extrusion deposition technique comprises preparing a material to be dispensed, such as preparing a powder in a hot melt extruder, and then feeding the material into a MED printing head. The MED printing head then dispenses the material to form the delayed sustained-release oral drug dosage form in an additive manner (layer-by-layer deposition). In some embodiments, each material of the drug dosage form, such as a first component and a swellable component, is dispensed from a different IVIED printing head. In some embodiments, the MED printing head dispenses the material according to instructions complied in one or more gcode files. Exemplary IVIED techniques are disclosed in, e.g., WO2019/137333, WO2018137686, and U.S. Pat. No. 10,201,503, each of which is incorporated herein by reference in its entirety.

In some embodiments, the 3D printing is carried out by fused deposition modeling (FDM). In some embodiments, the three-dimensional printing is carried out by melt extrusion deposition or hot melt extrusion coupled with a 3D printing technique, such as FDM. In some embodiments, the 3D printing is carried out by non-filament FDM. In some embodiments, the 3D printing is carried out by inkjet printing. In some embodiments, the 3D printing is carried out by selective laser sintering (SLS). In some embodiments, the 3D printing is carried out by stereolithography (SLA or SL). In some embodiments, the 3D printing is carried out by PolyJet, Multi-Jet Printing System (MJP), Perfactory, Solid Object Ultraviolet-Laser Printer, Bioplotter, 3D Bioprinting, Rapid Freeze Prototyping, Benchtop System, Selective Deposition Lamination (SDL), Laminated Objet Manufacturing (LOM), Ultrasonic Consolidation, ColorJet Printing (CJP), EOSINT Systems, Laser Engineered Net Shaping (LENS) and Aerosol Jet System, Electron Beam Melting (EBM), Laser CUSING®, Selective Laser Melting (SLM), Phenix PX™ Series, Microsintering, Digital Part Materialization (DPM), or VX System.

In some embodiments, the 3D printing methods described herein comprise a continuous feed method. In some embodiments, the 3D printing methods described herein comprise a batch feed method.

In some embodiments, the methods for producing the drug dosage forms described herein comprise a 3D printing technique, such as 3D printing in combination with another method, e.g., a combination of injection molding and 3D printing.

The method instructions for 3D printing a drug dosage form disclosed herein may be generated a variety of ways, including direct coding, derivation from a solid CAD model, or other means specific to the 3D printing machine's computer interface and application software. These instructions may include information on the number and spatial placement of droplets, and on general 3D print parameters such as the drop spacing in each linear dimension (X, Y, Z), and volume or mass of fluid per droplet. For a given set of materials, these parameters may be adjusted in order to refine the quality of structure created. The overall resolution of the structure created is a function of the powder particle size, the fluid droplet size, the print parameters, and the material properties.

In some embodiments, one or more components of the drug dosage form are created separately, such as printed separately, and later assembled to form the drug dosage form. In some embodiments, all components of the drug dosage form are created in a single method, such as printed in a single method, without requiring later assembly.

The drug dosage forms and components thereof described in the present application can be printed on a commercial scale. For example, in some embodiments, the methods disclosed herein may be used to 3D print 10,000 to 100,000 units of a drug dosage form per hour. In some embodiments, the methods disclosed herein may be used to 3D print 10,000 to 100,000 drug dosage forms per hour. In some embodiments, the methods disclosed herein may be used to 3D print 10,000 to 100,000 units of a dosage unit per hour. In some embodiments, the methods disclosed herein may be used to 3D print 10,000 to 100,000 dosage units per hour.

In some embodiments, the materials used to print the drug dosage forms and dosage units, or components thereof, e.g., precursor drug dosage forms, are each dispensed by a different printing head. For example, in some embodiments, a swelling agent of the swellable component is printed by a first printing head, a lid of the swellable component is printed by a second printing head, and a first component is printed by a third printing head.

The 3D printing methods described herein encompass printing the materials in any order that will allow for production of the oral drug dosage form and dosage units, or components thereof, e.g., precursor drug dosage forms, disclosed herein.

In some embodiments, the method for 3D printing comprises designing the drug dosage form or dosage unit, or component thereof, e.g., a precursor drug dosage form, in whole or in part, on a computer system. In some embodiments, the method comprises inputting parameters of the desired drug release profile and/or the oral drug dosage form and/or the dosage unit and/or a precursor drug dosage form into the computer system. In some embodiments, the method comprises providing one or more parameters to be printed, e.g., layer surface area, thickness, drug mass fraction, erosion rate. In some embodiments, the method comprises providing the desired drug release profile. In some embodiments, the methods comprise creating a virtual image of the item to be printed. In some embodiments, the method comprises creating a computer model that contains the pre-determined parameters. In some embodiments, the method comprises feeding the pre-determined parameters to a 3D printer and printing the item according to such pre-determined parameters. In some embodiments, the method comprises creating a 3D drawing of the item to be printed based on the pre-determined parameters, wherein the 3D drawing is created on a computer system. In some embodiments, the method comprises converting, such as slicing, a 3D drawing into 3D printing code, e.g., G code. In some embodiments, the method comprises using the computer system to execute 3D printing code, thereby printing according to the methods described herein.

### V. Methods of Delivering a Drug to an Individual

In some aspects, provided is a method of delivering a drug in an individual and allowing the drug to be retained in the stomach of the individual for an extended time, the method comprising orally administering to the individual a drug dosage form described herein. In some embodiments, the drug dosage form is configured to be retained in the stomach of an individual for an extended period of time (e.g., at least about 24 hours). In some embodiments, the drug can be release from the drug dosage form at a pre-determined time after administration of the drug dosage form to the individual. For example, in some embodiments, the drug is released in the stomach of the individual at least about 12 hours after administering the drug dosage form to the individual.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1

This example demonstrates the design and testing of a 3D printed drug dosage form configured for stomach retention. The drug dosage form comprised two swellable components, each comprising a swelling agent and an exterior lid, wherein the exterior lids formed opposing surfaces of the drug dosage form (FIGS. 1E and 1F). The exterior lids were configured to open outwardly after swelling of the swelling agent using a hinge mechanism. The swellable components were surround by a shell that formed a first component of the drug dosage form. Three configurations of the drug dosage form style were prepared and tested as follows.

### Material Preparation

Materials for printing the shell and the aspects of the swellable component were prepared as follows. Ingredients for printing were mixed according to a desired, pre-set ratio, added to a heated torque rheometer and melted together. The mixture was then removed from the torque rheometer and were ready to use.

The shell material and the exterior lid of the swelling component comprised EUDRAGIT® RSPO (RSPO). The swelling agent of the swellable component comprised ammonio methacrylate copolymer, polyethylene glycol molecular weight 400 (PEG400), and sodium carbonate or sodium alginate as shown in Table 1.

### Method of Printing

Three exemplary drug dosage forms (denoted A, B, and C) based on the design of FIGS. 1E and 1F were printed and tested for their swelling properties. To evaluate printing of the drug dosage forms, different printing fill rates were tested. The filling rate of the shell was set to 100%. The filling rate of the exterior lid was set to 100%, 50%, or 30%. The filling rate of the swelling agent was set to 100%, 50%, or 30%.

For each layer of each component, 3D printing was carried out in concentric circles, lines, or grids. Different materials, filling rates, and printing modes (in concentric circles, lines, or grids) may be combined.

The composition, filling mode, filling rate and dimensions of drug dosage form A, B, and C are shown in Table 1.

TABLE 1
Design of drug dosage forms A, B, and C.

| | | | Filling | Height | Width | Length |
|---|---|---|---|---|---|---|
| | | | rate of | of the | of the | of the |
| | | | the | swelling | exterior | exterior |
| | | Filling mode of | exterior | agent | lids | lids |
| Design | Composition | the shell material | lids | (mm) | (mm) | (mm) |
| A | MF:PEG400:Na₂CO₃ = 75:20:5 | 50%, in | 50% | 4.2 | 2 | 10 + 2.5 |
| | | concentric circles | | | | |
| B | MF:PEG400:sodium alginate = 7:2:1 | 100%, in | 33% | 4.2 | 2 | 10 + 2.5 |
| | | concentric circles | | | | |
| C | MF:PEG400:sodium alginate = 7:2:1 | 50%, in | 33% | 4.2 | 2 | 5 + 2.5 |
| | | concentric circles | | | | |

### Swelling Properties of Produced Drug Dosage Forms

Dosage forms A, B, and C were subjected to water and aqueous solution at pH 1.2 to test their swelling properties. Dimensions of A, B, and C were measured at 1 hour and 2 hours of swelling under both conditions and the results are reported in Table 2.

All three dosage forms showed significant increase in their heights at pH 1.2 and in water, with dosage form B having the largest increase under both conditions. The increases at pH 1.2 were lower than in water for dosage forms A and B, but were comparable for dosage form C under two conditions. These results demonstrated the potential of fine-tuning the size of the dosage forms after swelling by a combination of design choices (composition, filling rate, dimensions of the exterior lids, etc.), making it possible to achieve the desired swelling properties with high accuracy.

TABLE 2
Swelling measurements of drug dosage forms A, B, and C.

| | Height, | Height, | Height, | Height, | | | |
|---|---|---|---|---|---|---|---|
| | 1 h of | 1 h of | 2 h of | 2 h of | | | |
| | swelling | swelling | swelling | swelling | Dimensions | Dimensions 1 h of | Dimensions 2 h of |
| | (mm) in | (mm) at | (mm) in | (mm) at | before swelling | swelling in H₂O | swelling in H₂O |
| Design | H₂O | pH 1.2 | H₂O | pH 1.2 | (mm × mm × mm) | (mm × mm × mm) | (mm × mm × mm) |
| A | 12.282 | 9.33 | 14.62 | 11.94 | 21 × 11 × 6.3 | 21 × 11 × 12.28 | 21 × 11 × 14.62 |
| B | 13.24 | 12.7 | 15.44 | 14.81 | 21 × 11 × 6.3 | 21 × 11 × 13.24 | 21 × 11 × 15.44 |
| C | 12.31 | 12.39 | 13.32 | 13.43 | 21 × 11 × 6.3 | 21 × 11 × 12.31 | 21 × 11 × 13.32 |

What is claimed is:

1. A drug dosage form comprising a first component and a swellable component,
wherein the first component and the swellable component are connected,
wherein at least one of the first component and the swellable component comprises a drug,
wherein the swellable component, or a portion thereof, swells upon exposure to gastrointestinal fluid, and
wherein the swelling of the swellable component, or the portion thereof, increases the dimension of the drug dosage form, thereby allowing the drug to be retained in the stomach for at least about 24 hours.

2. The drug dosage form of claim 1, wherein the swellable component comprises an exterior lid and a swelling agent, wherein the exterior lid is connected to the swelling agent, and wherein the exterior lid is configured such that it forms at least a part of an exterior surface of the drug dosage form.

3. The drug dosage form of claim 2, wherein the surface of the exterior lid and a portion of the first component form an exterior surface of the drug dosage form.

4. The drug dosage form of claim 2 or 3, wherein a portion of the exterior lid is operably connected to the dosage form such that the exterior lid hinges outwardly from the drug dosage form due to swelling of the swellable component.

5. The drug dosage form of claim 2 or 3, wherein the exterior lid is configured such that swelling of the swellable component moves the entire exterior lid outwardly from the drug dosage form.

6. The drug dosage form of any one of claims 2-5, further comprising one or more swellable components each comprising an exterior lid.

7. The drug dosage form of claim 1, wherein the swellable component and the first component are configured as two separate layers.

8. The drug dosage form of claim 1, further comprising a second component, wherein the first component and the second component are connect, at least in part, via the swellable component.

9. The drug dosage form of claim 8, wherein the swelling of the swellable component shifts the relative positions of the first component and the second component, thereby increasing the dimension of the drug dosage form.

10. The drug dosage form of claim 8 or 9, wherein the first component has a first axis from a first proximal end to a first distal end, wherein the second component has a second axis from a second proximal end to a second distal end, and wherein the swellable component has a central axis, wherein upon swelling of the swellable component, the first axis and the second axis are shifted relative to each other.

11. The drug dosage form of claim 10, wherein the first axis and the second axis are aligned with each other without swelling of the swellable component, and rotate relative to each other upon swelling of the swellable component.

12. The oral dosage form of claim 11, wherein the first axis and the second axis become perpendicular to each other upon swelling of the swellable component.

13. The drug dosage of any one of claims 8-12, wherein at least a portion of the first component is configured to interface with the second component such that it creates a point of rotation.

14. The drug dosage form of claim 10, wherein the first axis and the second axis are aligned with each other, and are shifted away from the central axis upon swelling of the swellable component.

15. The drug dosage form of claim 14, wherein the drug dosage form further comprises a first guiding track and a second guiding track, wherein the upon swelling of the swellable component the first component shifts along with the first guiding track away from the central axis, and wherein the second component shifts along with the second guiding track away from the central axis.

16. The drug dosage form of claim 14 or 15, wherein the drug dosage form further comprises a third component, a third guiding track, a fourth component, and a fourth guiding track, wherein upon swelling of the swellable component the third component shifts along with the third guiding track away from the central axis, and the fourth component shifts along with the fourth guiding track away from the central axis.

17. The drug dosage form of any one of claims 1-16, wherein the first component comprises a first drug.

18. The drug dosage form of any one of claims 8-17, wherein the second component comprises a second drug.

19. The drug dosage form of any one of claims 15-18, wherein the third component comprises a third drug.

20. The drug dosage form of any one of claims 15-19, wherein the fourth component comprises a fourth drug.

21. The drug dosage form of any one of claims 1-20, wherein the swellable component comprises a core drug.

22. The drug dosage form of any one of claims 1-20, wherein the swellable component does not comprise a drug.

23. The drug dosage form of any one of claims 17-21, wherein at least two of the first drug, the second drug, the third drug, the fourth drug, and the core drug are the same.

24. The drug dosage form of any one of claims 17-21, wherein at least two of the first drug, the second drug, the third drug, the fourth drug, and the core drug are the different from each other.

25. The drug dosage form of any one of claims 17-21, wherein at least one of the first drug, the second drug, the third drug, the fourth drug, and the core drug is poorly water soluble.

26. The drug dosage form of any one of claims 17-21 and 23-25, wherein any one of the first drug, the second drug, the third drug, the fourth drug, and the core drug is in a compartment embedded in a substrate material.

27. The drug dosage form of claim 26, wherein the first component, the second component, the third component, and/or the fourth component comprises a drug contained within a compartment.

28. The drug dosage form of claim 22, wherein the compartment comprises a plug.

29. The drug dosage form of claim 27 or 28, wherein the first component, the second component, the third component, and/or the fourth component comprises more than one drug-filled compartment.

30. The drug dosage form of any one of claims 1-29, wherein the drug dosage form is an oral drug dosage form.

31. A commercial batch of a drug dosage form of any one of claims 1-30, wherein the commercial batch has a standard deviation of about 0.05 or less for each of the following:
an amount of a drug in the drug dosage form;
weight of the drug dosage form;
a largest crossing dimension of the oral drug dosage form;
a crossing dimension perpendicular to the largest crossing dimension of the oral drug dosage form;
a largest crossing dimension of the oral drug dosage form after swelling of the swelling component; and
a crossing dimension perpendicular to the largest crossing dimension of the oral drug dosage form after swelling of the swelling component.

32. The commercial batch of claim 31, wherein the commercial batch comprises at least about 1000 of the drug dosage forms.

33. The drug dosage form of any one of claims 1-30, wherein the drug dosage form is made by a 3D printing technique.

34. A method of three-dimensional (3D) printing of a drug dosage form of any one of claims 1-30, the method comprising:
(a) dispensing the first component, or a portion thereof; and
(b) dispensing the swellable component, or a portion thereof.

35. The method of claim 34, wherein the dispensing is via melt extrusion deposition (MED).

36. The method of claim 34 or 35, wherein dispensing of the first component, or a portion thereof, and dispensing of the swellable component, or a portion thereof, are performed by different printing heads.

37. A method for preparing a drug dosage form by three-dimensional (3D) printing,
wherein the drug dosage form comprises a first component, a second component, and a swellable component,
the method comprising:
(a) dispensing a material of the first component;
(b) dispensing a material of the second component; and
(c) dispensing a material of the swellable component.

38. The method of claim 37, wherein the dispensing is via melt extrusion deposition (IVIED).

39. The method of claim 37 or 38, wherein dispensing of each material is performed by a different printing head.

40. The method of any one of claims 34-39, wherein the drug dosage form is 3D printed using a layer-by-layer technique.

41. A method of delivering a drug in an individual and allowing the drug to be retained in the stomach of the individual for an extended time, the method comprising orally administering to the individual a drug dosage form of any one of claims 1-30.
